(19) United States
(12) Patent Application Publication
(10) Pub. No.: US 2024/0124532 A1
(43) Pub. Date: Apr. 18, 2024

(54) CHLAMYDIA TRACHOMATIS ANTIGENIC POLYPEPTIDES AND USES THEREOF FOR VACCINE PURPOSES

(71) Applicants: Institut National de la Santé et de la Recherche Médicale (INSERM), Paris (FR); Assistance Publique - Hopitaux de Paris, Paris (FR); Universite Paris Est Creteil Val De Marne, Creteil (FR); Baylor Research Institute, Dallas, TX (US)

(72) Inventors: Yves LEVY, Creteil Cedex (FR); Sandra ZURAWSKI, Dallas, TX (US); Gérard ZURAWSKI, Dallas, TX (US); Mireille CENTLIVRE, Creteil Cedex (FR); Lydie DIEUDONNE, Creteil Cedex (FR); Sylvain CARDINAUD, Creteil Cedex (FR)

(73) Assignees: Institut National de la Santé et de la Recherche Médicale (INSERM), Paris (FR); Assistance Publique - Hopitaux de Paris, Paris (FR); Universite Paris Est Creteil Val De Marne, Creteil (FR); Baylor Research Institute, Dallas, TX (US)

(21) Appl. No.: 18/274,848

(22) PCT Filed: Jan. 28, 2022
(86) PCT No.: PCT/EP2022/052104

§ 371 Date: Jul. 28, 2023

(30) Foreign Application Priority Data
Jan. 29, 2021 (EP) 21305118.8

(51) Int. Cl. C07K 14/295; A61K 39/118; C07K 16/28

(52) U.S. Cl. CPC C07K 14/295; A61K 39/118; C07K 16/2878; C07K 2319/33; C07K 2319/40

(57) ABSTRACT

Chlamydiae are intracellular bacterial pathogens responsible for a variety of infections. The inventors have set up candidate vaccines against Chlamydia trachomatis. In particular, the inventors have identified specific epitopes to be included in vaccine candidates thanks to in silico analysis of the amino-acid sequence of these proteins to map predicted MHC-I and -II epitopes by online software (NetMHC-4.0 and NetMHCII-2.3) and peptide binding prediction software. B cell epitopes were also mapped using online software (BepiPred-2.0 and Discotope). Finally, the inventors have generated some specific CD40 or Langerin antibodies comprising one or more identified epitope(s) of the present invention and that are suitable for vaccine purposes. Therefore, the present invention relates to Chlamydia trachomatis (Ct) antigenic polypeptides and uses thereof for vaccine purposes.

### FIELD OF THE INVENTION

The present invention is in the field of medicine, in particular vaccinology.

### BACKGROUND OF THE INVENTION

Chlamydiae are intracellular bacterial pathogens responsible for a variety of infections. For instance, Chlamydia trachomatis is the causative agent of human sexually transmitted disease and eye infections (Trachoma). Worldwide, it is estimated that 92 million individuals become sexually infected with Chlamydia trachomatis. Urogenital infections with Chlamydia trachomatis are of public health concern because of its high prevalence and the fact that it's a risk factor for ectopic pregnancy and infertility. In addition to this Chlamydia trachomatis infections have been shown to facilitate the transmission of HIV and act as a co-factor in HPV-induced cervical carcinoma. The duration of untreated genital Chlamydia trachomatis infection can be prolonged, and complete clearance is often not reached within the first 12 months. From human studies it is known that some degree of protective immunity against genital re-infection develops, although it appears at best to be partial. The infection is effectively controlled by antibiotic therapy; however the high prevalence of asymptomatic cases suggests that sustainable disease control can only be envisaged if an effective Chlamydia vaccine is developed.

MOMP is highly immunogenic in humans and animals and has therefore been studied in great detail as a vaccine candidate, both as a natively purified protein, recombinantly and as DNA-vaccine. Mainly VS4 has attracted interest as an immunogen because this region was shown to contain a highly conserved species-specific epitope embedded in the variable region. Importantly, this conserved epitope in the VS4 region can elicit a broadly cross-reactive immune response, which is able to neutralize multiple serovars, among them the most prevalent D, E and F. Reasons for the lack of protection when using the MOMP peptides can be numerous; including the less efficient targeting of the antigen presenting cells but most likely reflects that the vaccine molecule is not sufficiently immunogenic for use as a vaccine.

### SUMMARY OF THE INVENTION

The present invention is defined by the claims. In particular the present invention relates to Chlamydia trachomatis (Ct) antigenic polypeptides and uses thereof for vaccine purposes.

### DETAILED DESCRIPTION OF THE INVENTION

### Definitions

As used herein, the term “subject” or “subject in need thereof”, is intended for a human or non-human mammal. Typically the patient is affected or likely to be infected with Chlamydia trachomatis.

As used herein, the term “Chlamydia trachomatis” has its general meaning in the art and refers to a bacterium that is the causative agent of human sexually transmitted disease and eye infections (Trachoma), which can manifest in various ways, including: trachoma, lymphogranuloma venereum, nongonococcal urethritis, cervicitis, salpingitis, pelvic inflammatory disease. Chlamydia trachomatis is the most common infectious cause of blindness and the most common sexually transmitted bacterium.

As used herein, the term “asymptomatic” refers to a subject who experiences no detectable symptoms for the chlamydia infection. As used herein, the term “symptomatic” refers to a subject who experiences detectable symptoms of chlamydia infection. Symptoms of chlamydia infection include but are not limited to pain when urinating, unusual vaginal discharge, pain in the tummy or pelvis, pain during sex, bleeding after sex, bleeding between periods for women as well as pain when urinating, white, cloudy or watery discharge from the tip of the penis, burning or itching in the urethra (the tube that carries urine out of the body), or pain in the testicles for men.

As used herein, the terms “polypeptide”, “peptide”, and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides when discussed in the context of gene therapy refer to the respective intact polypeptide, or any fragment or genetically engineered derivative thereof, which retains the desired biochemical function of the intact protein.

As used herein, the term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

As used herein, the expression “derived from” refers to a process whereby a first component (e.g., a first polypeptide), or information from that first component, is used to isolate, derive or make a different second component (e.g., a second polypeptide that is different from the first one).

As used herein, the term “encoding” refers to the inherent property of specific sequences of nucleotides in a polynucleotide, such as, for example, a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase “nucleotide sequence that encodes a protein or a RNA” may also include introns to the extent that the nucleotide sequence encoding the protein may in some version contain an intron(s).

As used herein, the terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence.

As used herein, the term “promoter/regulatory sequence” refers to a nucleic acid sequence (such as, for example, a DNA sequence) recognized by the synthetic machinery of the cell, or introduced synthetic machinery, required to initiate the specific transcription of a polynucleotide sequence, thereby allowing the expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

As used herein, the term “transformation” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA bas been “transformed”.

As used herein, the term “expression system” means a host cell and compatible vector under suitable conditions, e.g., for the expression of a protein coded for by foreign DNA carried by the vector and introduced to the host cell.

As used herein, the “percent identity” between the two sequences is a function of the number of identical positions shared by the sequences (i.e., % identity=number of identical positions/total number of positions×100), taking into account the number of gaps, and the length of each gap, which need to be introduced for optimal alignment of the two sequences.

The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification. According to the invention a first amino acid sequence having at least 80% of identity with a second amino acid sequence means that the first sequence has 80, 81, 82, 83, 84, 85, 86; 87; 88; 89; 90; 91; 92; 93; 94; 95; 96; 97; 98; 99 or 100% of identity with the second amino acid sequence. According to the invention a first amino acid sequence having at least 90% of identity with a second amino acid sequence means that the first sequence has 90; 91; 92; 93; 94; 95; 96; 97; 98; 99 or 100% of identity with the second amino acid sequence.

As used herein, the term “conjugate” or interchangeably “conjugated polypeptide” is intended to indicate a composite or chimeric molecule formed by the covalent attachment of one or more polypeptides. The term “covalent attachment” “or “conjugation” means that the polypeptide and the non-peptide moiety are either directly covalently joined to one another, or else are indirectly covalently joined to one another through an intervening moiety or moieties, such as a bridge, spacer, or linkage moiety or moieties. A particular conjugate is a fusion protein.

As used herein, the term “fusion protein” indicates a protein created through the attaching of two or more polypeptides which originated from separate proteins. In particular fusion proteins can be created by recombinant DNA technology and are typically used in biological research or therapeutics. Fusion proteins can also be created through chemical covalent conjugation with or without a linker between the polypeptides portion of the fusion proteins. In the fusion protein the two or more polypeptide are fused directly or via a linker.

As used herein, the term “directly” means that the first amino acid at the N-terminal end of a first polypeptide is fused to the last amino acid at the C-terminal end of a second polypeptide. This direct fusion can occur naturally as described in (Vigneron et al., Science 2004, PMID 15001714), (Warren et al., Science 2006, PMID 16960008), (Berkers et al., J. Immunol. 2015a, PMID 26401000), (Berkers et al., J. Immunol. 2015b, PMID 26401003), (Delong et al., Science 2016, PMID 26912858) (Liepe et al., Science 2016, PMID 27846572), (Babon et al., Nat. Med. 2016, PMID 27798614).

As used herein, the term “linker” has its general meaning in the art and refers to an amino acid sequence of a length sufficient to ensure that the proteins form proper secondary and tertiary structures. In some embodiments, the linker is a peptidic linker which comprises at least one, but less than 30 amino acids e.g., a peptidic linker of 2-30 amino acids, preferably of 10-30 amino acids, more preferably of 15-30 amino acids, still more preferably of 19-27 amino acids, most preferably of 20-26 amino acids. In some embodiments, the linker has 2; 3; 4; 5; 6; 7; 8; 9; 10; 11; 12; 13; 14; 15; 16; 17; 18; 19; 20; 21; 22; 23; 24; 25; 26; 27; 28; 29; 30 amino acid residues. Typically, linkers are those which allow the compound to adopt a proper conformation. The most suitable linker sequences (1) will adopt a flexible extended conformation, (2) will not exhibit a propensity for developing ordered secondary structure which could interact with the functional domains of fusion proteins, and (3) will have minimal hydrophobic or charged character which could promote interaction with the functional protein domains.

As used herein, the term “antibody” refers to immunoglobulin molecules and immunologically active portions of immunoglobulin molecules, i.e., molecules that contain an antigen binding site that immunospecifically binds to an antigen. In natural antibodies of rodents and primates, two heavy chains are linked to each other by disulfide bonds, and each heavy chain is linked to a light chain by a disulfide bond. There are two types of light chains, lambda (1) and kappa (κ). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) can participate in the antibody binding site, or influence the overall domain structure and hence the combining site. Complementarity Determining Regions or CDRs refer to amino acid sequences that together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system. For the agonist antibodies described hereafter, the CDRs have been determined using CDR finding algorithms from www.bioinf.org.uk—see the section entitled «How to identify the CDRs by looking at a sequence» within the Antibodies pages.

As used herein, the term “immunoglobulin domain” refers to a globular region of an antibody chain (such as e.g. a chain of a heavy chain antibody or a light chain), or to a polypeptide that essentially consists of such a globular region.

As used herein, the term “Fc region” is used to define the C-terminal region of an immunoglobulin heavy chain, including native sequence Fc region and variant Fc regions. The human IgG heavy chain Fc region is generally defined as comprising the amino acid residue from position C226 or from P230 to the carboxyl-terminus of the IgG antibody. The numbering of residues in the Fc region is that of the EU index of Kabat. The C-terminal lysine (residue K447) of the Fc region may be removed, for example, during production or purification of the antibody. Accordingly, a composition of antibodies of the invention may comprise antibody populations with all K447 residues removed, antibody populations with no K447 residues removed, and antibody populations having a mixture of antibodies with and without the K447 residue.

As used herein, the term “chimeric antibody” refers to an antibody which comprises a VH domain and a VL domain of a non-human antibody, and a CH domain and a CL domain of a human antibody. In some embodiments, a “chimeric antibody” is an antibody molecule in which (a) the constant region (i.e., the heavy and/or light chain), or a portion thereof, is altered, replaced or exchanged so that the antigen binding site (variable region) is linked to a constant region of a different or altered class, effector function and/or species, or an entirely different molecule which confers new properties to the chimeric antibody.; or (b) the variable region, or a portion thereof, is altered, replaced or exchanged with a variable region having a different or altered antigen specificity. Chimeric antibodies also include primatized and in particular humanized antibodies. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). (see U.S. Pat. No. 4,816,567; and Morrison et al., Proc. Natl. Acad. Sci. USA, 81:6851-6855 (1984)).

As used herein, the term “humanized antibody” include antibodies which have the 6 CDRs of a murine antibody, but humanized framework and constant regions. More specifically, the term “humanized antibody”, as used herein, may include antibodies in which CDR sequences derived from the germline of another mammalian species, such as a mouse, have been grafted onto human framework sequences.

As used herein the term “human monoclonal antibody”, is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). However, in one embodiment, the term “human monoclonal antibody”, as used herein, is not intended to include antibodies in which CDR sequences derived from the germline of another mammalian species, such as a mouse, have been grafted onto human framework sequences.

As used herein, the term “immune response” refers to a reaction of the immune system to an antigen in the body of a host, which includes generation of an antigen-specific antibody and/or cellular cytotoxic response. The immune response to an initial antigenic exposure (primary immune response) is typically, detectable after a lag period of from several days to two weeks; the immune response to subsequent stimulus (secondary immune response) by the same antigen is more rapid than in the case of the primary immune response. An immune response to a transgene product may include both humoral (e.g., antibody response) and cellular (e.g., cytolytic T cell response) immune responses that may be elicited to an immunogenic product encoded by the transgene. The level of the immune response can be measured by methods known in the art (e.g., by measuring antibody titre).

As used herein the term “APCs” or “Antigen Presenting Cells” denotes cells that are capable of activating T-cells, and include, but are not limited to, certain macrophages, B cells and dendritic cells.

As used herein, the term “Dendritic cells” or “DCs” refer to any member of a diverse population of morphologically similar cell types found in lymphoid or non-lymphoid tissues. These cells are characterized by their distinctive morphology, high levels of surface MHC-class II expression (Steinman, et al., Ann. Rev. Immunol. 9:271 (1991); incorporated herein by reference for its description of such cells).

As used herein, the term “CD40” has its general meaning in the art and refers to human CD40 polypeptide receptor. In some embodiments, CD40 is the isoform of the human canonical sequence as reported by UniProtKB-P25942 (also referred as human TNR5).

As used herein, the term “CD40L” has its general meaning in the art and refers to human CD40L polypeptide, for example, as reported by UniProtKB-P25942, including its CD40-binding domain of SEQ ID NO:1. CD40L may be expressed as a soluble polypeptide and is the natural ligand of CD40 receptor.

> CD40L binding domain
SEQ ID NO: 1
MQKGDQNPQIAAHVISEASSKTTSVLQWAEKGYYTMSNNLVTLENGKQLT
VKRQGLYYIYAQVTFCSNREASSQAPFIASLCLKSPGRFERILLRAANTH
SSAKPCGQQSIHLGGVFELQPGASVFVNVTDPSQVSHGTGFTSFGLLKL

As used herein, the term “CD40 agonist antibody” is intended to refer to an antibody that increases CD40 mediated signaling activity in the absence of CD40L in a cell-based assay, such as the B cell proliferation assay. In particular, the CD40 agonist antibody (i) it induces the proliferation of B cell, as measured in vitro by flow cytometric analysis, or by analysis of replicative dilution of CFSE-labeled cells; and/or (ii) induces the secretion of cytokines, such as IL-6, IL-12, or IL-15, as measured in vitro with a dendritic cell activation assay.

As used herein, the term “Langerin” has its general meaning in the art and refers to human C-type lectin domain family 4 member K polypeptide. In some embodiments, Langerin is the isoform of the human canonical sequence as reported by UniProtKB-Q9UJ71 (also referred as human CD207).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical-Sciences, Sixteenth Edition, E. W. Martin (Mack Publishing Co., Easton, Pa., 1980) discloses various carriers used in formulating pharmaceutical compositions and known techniques for the preparation thereof.

As used herein, the term “vaccination” or “vaccinating” means, but is not limited to, a process to elicit an immune response in a subject against a particular antigen.

As used herein, the term “vaccine composition” is intended to mean a composition which can be administered to humans or to animals in order to induce an immune system response; this immune system response can result in the activation of certain cells, in particular APCs, T lymphocytes and B lymphocytes.

As used herein the term “antigen” refers to a molecule capable of being specifically bound by an antibody or by a T cell receptor (TCR) if processed and presented by MHIC molecules. An antigen is additionally capable of being recognized by the immune system and/or being capable of inducing a humoral immune response and/or cellular immune response leading to the activation of B- and/or T-lymphocytes. An antigen can have one or more epitopes or antigenic sites (B- and T-epitopes).

As used herein, the term “adjuvant” refers to a compound that can induce and/or enhance the immune response against an antigen when administered to a subject or an animal. It is also intended to mean a substance that acts generally to accelerate, prolong, or enhance the quality of specific immune responses to a specific antigen. In the context of the present invention, the term “adjuvant” means a compound, which enhances both innate immune response by affecting the transient reaction of the innate immune response and the more long-lived effects of the adaptive immune response by activation and maturation of the antigen-presenting cells (APCs) especially Dendritic cells (DCs).

As used herein, the expression “therapeutically effective amount” is meant a sufficient amount of the active ingredient of the present invention to induce an immune response at a reasonable benefit/risk ratio applicable to the medical treatment.

Ct Antigenic Polypeptides of the Present Invention:

The first object of the present invention relates to a Chlamydia trachomatis (Ct) antigenic polypeptide that comprises:
a VS4 bis polypeptide having an amino acid sequence having at least 90% of identity with the amino acid as set forth in SEQ ID NO:2, or
a YchM polypeptide having an amino acid sequence having at least 90% of identity with the amino acid as set forth in SEQ ID NO:3, or
a PgP3 polypeptide having an amino acid sequence having at least 90% of identity with the amino acid as set forth in SEQ ID NO:4, or
a PmPG polypeptide having an amino acid sequence having at least 90% of identity with the amino acid as set forth in SEQ ID NO:5, or
a NqrC polypeptide having an amino acid sequence having at least 90% of identity with the amino acid as set forth in SEQ ID NO:6.

| | VS4 bis |
|---|---|
| SEQ ID NO: 2 | |
| | ASIDYHEWQASLALSYRLNMFTPYIGVKWS |
| | YchM antigen |
| SEQ ID NO: 3 | |
| | EKPPKIFILCMTRVPTIDASAMHALEEFFL |
| | PgP3 antigen |
| SEQ ID NO: 4 | |
| | GKFTVTPKSSGSMFLVSADIIASRMEGGVV |
| | PmPG antigen |
| SEQ ID NO: 5 | |
| | PAANQLITLSNLHLSLSSLLANNAVTNPPT |
| | NqrC antigen |
| SEQ ID NO: 6 | |
| | CNGVTESFSHSLAPYRALLTFFANSKPSGE |

In some embodiment, the Chlamydia trachomatis (Ct) antigenic polypeptide comprises or consist of:
a VS4 bis polypeptide having an amino acid sequence as set forth in SEQ ID NO:2, or
a YchM polypeptide having an amino acid as set forth in SEQ ID NO:3, or
a PgP3 polypeptide having an amino acid as set forth in SEQ ID NO:4, or
a PmPG polypeptide having an amino acid as set forth in SEQ ID NO: 5, or
a NqrC polypeptide having an amino acid as set forth in SEQ ID NO: 6.

Ct Antigenic Fusion Protein of the Present Invention

A further object of the present invention relates to a Ct antigenic fusion protein that comprises one or more Ct antigenic polypeptide(s) of the present invention;

In some embodiments, the Ct antigenic fusion protein of the present invention comprises two or more Ct antigenic polypeptide(s) of the present invention.

In some embodiments, the Ct antigenic fusion protein of the present invention comprises 1, 2, 3, 4, or 5 Ct antigenic polypeptides of the present invention.

In some embodiments, the Ct antigenic polypeptides are fused to each other directly or via a linker.

In some embodiments, the linker is selected from the group consisting of FlexV1, f1, f2, f3, or f4 as described below.

In some embodiments, the linker is selected from the group consisting of SEQ ID NO:7 (FlexV1), SEQ ID NO:8 (f1), SEQ ID NO:9 (f2), SEQ ID NO:10 (f3) and SEQ ID NO:11(f4).

(flexV1, SEQ ID NO: 7)
QTPTNTISVTPTNNSTPTNNSNPKPNP
(f1, SEQ ID NO: 8)
SSVSPTTSVHPTPTSVPPTPTKSSP
(f2, SEQ ID NO: 9)
PTSTPADSSTITPTATPTATPTIKG
(f3, SEQ ID NO: 10)
TVTPTATATPSAIVTTITPTATTKP
(f4, SEQ ID NO: 11)
TNGSITVAATAPTVTPTVNATPSAA

In some embodiments, the Ct antigenic fusion protein of the present invention has the formula of (Ag-L)n wherein Ag represents a Ct antigenic polypeptide of the present invention, L represents a linker of the present invention and n represents an integer number from 1 to 5.

In some embodiments, the fusion protein comprises in said order the VS4 Bis antigenic polypeptide, the PmpG antigenic polypeptide, the PgP3 antigenic polypeptide, the YchM antigenic polypeptide and the NqrC antigenic polypeptide.

In some embodiments, the Ct antigenic fusion protein of the present invention has the formula of VS4 bis-f1-PmpG-f2-PgP3-f3-YchM-f4-NqrC and that consists of the amino acid sequence as set forth in SEQ ID NO:12.

> fusion protein VS4 bis-f1-PmpG-f2-
PgP3-f3-YchM-f4-NqrC
SEQ ID NO: 12
ASIDYHEWQASLALSYRLNMFTPYIGVKWSSSVSPTTSVHPTPTSVPPTP
TKSSPPAANQLITLSNLHLSLSSLLANNAVTNPPTPTSTPADSSTITPTA
TPTATPTIKGGKFTVTPKSSGSMFLVSADIIASRMEGGVVTVTPTATATP
SAIVTTITPTATTKPEKPPKIFILCMTRVPTIDASAMHALEEFFLINGSI
TVAATAPTVTPTVNATPSAACNGVTESFSHSLAPYRALLTFFANSKPSGE

Methods of Producing the Ct Antigenic Polypeptides and Fusion Proteins of the Present Invention:

The Ct antigenic polypeptides and Ct antigenic fusion proteins of the invention may be produced by any technique known per se in the art, such as, without limitation, any chemical, biological, genetic or enzymatic technique, either alone or in combination. Knowing the amino acid sequence of the desired sequence, one skilled in the art can readily produce said polypeptides, by standard techniques for production of polypeptides. For instance, they can be synthesized using well-known solid phase method, preferably using a commercially available peptide synthesis apparatus (such as that made by Applied Biosystems, Foster City, California) and following the manufacturer's instructions. Alternatively, the polypeptides and fusions proteins of the invention can be synthesized by recombinant DNA techniques as is now well-known in the art. For example, these fragments can be obtained as DNA expression products after incorporation of DNA sequences encoding the desired (poly) peptide into expression vectors and introduction of such vectors into suitable eukaryotic or prokaryotic hosts that will express the desired polypeptide, from which they can be later isolated using well-known techniques.

Antibodies of the Present Invention:

A further object of the present invention relates to an antibody that is directed against a surface antigen of an antigen presenting cell wherein the heavy chain and/or the light chain is conjugated or fused to a Ct antigenic fusion protein of the present invention.

In some embodiments, the heavy chain of the antibody is conjugated or fused to the Ct antigenic fusion protein of the present invention.

In some embodiments, the light chain of the antibody is conjugated or fused to the Ct antigenic fusion protein of the present invention.

In some embodiments, both the heavy and light chains of the antibody are conjugated or fused to the Ct antigenic fusion protein of the present invention.

In some embodiments, the antibody is an IgG antibody, preferably of an IgG1 or IgG4 antibody, or even more preferably of an IgG4 antibody.

In some embodiments, the antibody is a chimeric antibody, in particular a chimeric mouse/human antibody.

In some embodiments, the antibody is humanized antibody.

Chimeric or humanized antibodies can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al.

In some embodiments, the antibody is a human antibody. In some embodiments, human antibodies can be identified using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.” The HuMAb Mouse® (Medarex, Inc.) contains human immunoglobulin gene miniloci that encode un-rearranged human heavy (p and y) and x light chain immunoglobulin sequences, together with targeted mutations that inactivate the endogenous p and K chain loci (see e.g., Lonberg, et al., 1994 Nature 368(6474): 856-859). In some embodiments, human antibodies can be raised using a mouse that carries human immunoglobulin sequences on transgenes and transchomosomes such as a mouse that carries a human heavy chain transgene and a human light chain transchromosome. Such mice, referred to herein as “KM mice”, are described in detail in PCT Publication WO 02/43478 to Ishida et al.

In some embodiments, the antibody is specific for a cell surface marker of a professional APC. The antibody may be specific for a cell surface marker of another professional APC, such as a B cell or a macrophage.

In some embodiments, the antibody is selected from an antibody that specifically binds to DC immunoreceptor (DCIR), MHC class I, MHC class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-7 receptor and IL-2 receptor, ICAM-1, Fey receptor, LOX-1, and ASPGR.

In some embodiments, the antibody is specific for CD40.

In some embodiments, the anti-CD40 antibody derives from the 12E12 antibody and comprises:
a heavy chain comprising the complementarity determining regions CDR1H, CDR2H and CDR3H, the CDR1H having the amino acid sequence GFTFSDYYMY (SEQ ID NO:13), the CDR2H having the amino acid sequence YINSGGGSTYYPDTVKG (SEQ ID NO:14), and the CDR3H having the amino acid sequence RGLPFHAMDY (SEQ ID NO:15),
and a light chain comprising the complementarity determining regions CDR1L, CDR2L and CDR3L, the CDR1L having the amino acid sequence SASQGISNYLN (SEQ ID NO:16) the CDR2L having the amino acid sequence YTSILHS (SEQ ID NO:17) and the CDR3L having the amino acid sequence QQFNKLPPT (SEQ ID NO:18).

In some embodiments, the anti-CD40 antibody derives from the 11B6 antibody and comprises:
a heavy chain comprising the complementarity determining regions CDR1H, CDR2H and CDR3H, the CDR1H having the amino acid sequence GYSFTGYYMH (SEQ ID NO:19), the CDR2H having the amino acid sequence RINPYNGATSYNQNFKD (SEQ ID NO:20), and the CDR3H having the amino acid sequence EDYVY (SEQ ID NO:21), and
a light chain comprising the complementarity determining regions CDR1L, CDR2L and CDR3L, the CDR1L having the amino acid sequence RSSQSLVHSNGNTYLH (SEQ ID NO:22) the CDR2L having the amino acid sequence KVSNRFS (SEQ ID NO:23) and the CDR3L having the amino acid sequence SQSTHVPWT (SEQ ID NO:24).

In some embodiments, the anti-CD40 antibody derives from the 12B4 antibody and comprises:
a heavy chain comprising the complementarity determining regions CDR1H, CDR2H and CDR3H, the CDR1H having the amino acid sequence GYTFTDYVLH (SEQ ID NO:25), the CDR2H having the amino acid sequence YINPYNDGTKYNEKFKG (SEQ ID NO:26), and the CDR3H having the amino acid sequence GYPAYSGYAMDY (SEQ ID NO:27), and
a light chain comprising the complementarity determining regions CDR1L, CDR2L and CDR3L, the CDR1L having the amino acid sequence RASQDISNYLN (SEQ ID NO:28) the CDR2L having the amino acid sequence YTSRLHS (SEQ ID NO:29) and the CDR3L having the amino acid sequence HHGNTLPWT (SEQ ID NO:30).

In some embodiments, the anti-CD40 antibody is selected from the group consisting of selected mAb1, mAb2, mAb3, mAb4, mAb5 and mAb6 as described in Table A.

In some embodiments, the anti-CD40 antibody is selected from the group consisting of:
an anti-CD40 antibody comprising a heavy chain having an amino sequence as set forth in SEQ ID NO:31 and a light chain having an amino sequence as set forth in SEQ ID NO:32 (mAb1);
an anti-CD40 antibody comprising a heavy chain having an amino sequence as set forth in SEQ ID NO:33 and a light chain having an amino sequence as set forth in SEQ ID NO:32 (mAb2);
an anti-CD40 antibody comprising a heavy chain having an amino sequence as set forth in SEQ ID NO:34 and a light chain having an amino sequence as set forth in SEQ ID NO:35 (mAb3);
an anti-CD40 antibody comprising a heavy chain having an amino sequence as set forth in SEQ ID NO:36 and a light chain having an amino sequence as set forth in SEQ ID NO:37 (mAb4);
an anti-CD40 antibody comprising a heavy chain having an amino sequence as set forth in SEQ ID NO:38 and a light chain having an amino sequence as set forth in SEQ ID NO:39 (mAb5); and
an anti-CD40 antibody comprising a heavy chain having an amino sequence as set forth in SEQ ID NO:40 and a light chain having an amino sequence as set forth in SEQ ID NO:41 (mAb6).

TABLE A
CD40 antibodies

| | | |
|---|---|---|
| mAb1 [11B6 VH/VkV2] | SEQ ID NO: 31 | SEQ ID NO: 32 |
| mAb2 [11B6 VHV3/VkV2] | SEQ ID NO: 33 | SEQ ID NO: 32 |
| mAb3 [12B4] | SEQ ID NO: 34 | SEQ ID NO: 35 |
| mAb4 [24A3] | SEQ ID NO: 36 | SEQ ID NO: 37 |
| mAb5 [CP870,893] | SEQ ID NO: 38 | SEQ ID NO: 39 |
| mAb6 [12E12] | SEQ ID NO: 40 | SEQ ID NO: 41 |

SEQ ID NO: 31 (Amino acid sequence of variable heavy chain region (VH)
(v2) of Humanized 11B6)
EVQLVQSGAEVKKPGASVKISCKASGYSFTGYYMHWVKQAHGQGLEWIGRINPYNGATSYNONFKDRATL
TVDKSTSTAYMELSSLRSEDTAVYYCAREDYVYWGQGTTVTVSSAS
SEQ ID NO: 32 (Amino acid sequence of variable light chain (VL) Vk
(v2) of humanized 11B6 VL)
DVVMTQSPLSLPVTLGQPASISCRSSQSLVHSNGNTYLHWYQQRPGQSPRLLIYKVSNRFSGVPDRESGS
GSGTDFTLKISRVEAEDVGVYFCSQSTHVPWTFGGGTK
SEQ ID NO: 33 (Amino acid sequence of variable heavy chain region VH
(v3) of humanized 11B6)
EVQLVQSGAEVKKPGASVKVSCKASGYSFTGYYMHWVRQAPGOGLEWIGRINPYNGATSYNONFKDRVTL
TVDKSTSTAYMELSSLRSEDTAVYYCAREDYVYWGQGTTVTVSSAS
SEQ ID NO: 34 (VH amino acid sequence of mAb3 (12B4))
EVQLQQSGPELVKPGASVKMSCKASGYTFTDYVLHWVKQKPGQGLEWIGYINPYNDGTKYNEKFKGKATL
TSDKSSSTAYMELSSLTSEDSAVYYCARGYPAYSGYAMDYWGQGTSVTVSSAS
SEQ ID NO: 35 (VL amino acid sequence of mAb3 (12B4))
DIQMTQTTSSLSASLGDRVTISCRASQDISNYLNWYQQKPDGTVKLLIYYTSRLHSGVPSRFSGSGSGTD
YSLTISNLEQEDIATYFCHHGNTLPWTFGGGTK
SEQ ID NO: 36 (VH amino acid sequence of mAb4 (24A3 HC))
DVQLQESGPDLVKPSQSLSLTCTVTGYSITSDYSWHWIRQFPGNKLEWMGYIYYSGSTNYNPSLKSRISI
TRDTSKNQFFLQLNSVTTEDSATYFCARFYYGYSFFDYWGQGTTLTVSSAS
SEQ ID NO: 37 (VL amino acid sequence of mAb4 (24A3 KC))
QIVLTQSPAFMSASPGEKVTMTCSASSSVSYMHWYQQKSGTSPKRWIYDTSKLASGVPARFSGSGSGTSY
SLTISSMEAEDAATYYCQQWSSNPLTFGAGTK
SEQ ID NO: 38 (VH amino acid sequence of mAb5)
QVQLVQSGAEVKKPGASVKVSCKASGYTFTGYYMHWVRQAPGQGLEWMGWINPDSGGTNYAQKFQGRVTM
TRDTSISTAYMELNRLRSDDTAVYYCARDQPLGYCTNGVCSYFDYWGQGTLVTVSSAS
SEQ ID NO: 39 (VL amino acid sequence of mAb5)
DIQMTQSPSSVSASVGDRVTITCRASQGIYSWLAWYQQKPGKAPNLLIYTASTLQSGVPSRFSGSGSGTD
FTLTISSLOPEDFATYYCQQANIFPLTFGGGTK
SEQ ID NO: 40 (VH amino acid sequence of mAb6 (12E12 H3 Humanized HC))
EVQLVESGGGLVQPGGSLKLSCATSGFTFSDYYMYWVRQAPGKGLEWVAYINSGGGSTYYPDTVKGRFTI
SRDNAKNTLYLOMNSLRAEDTAVYYCARRGLPFHAMDYWGQGTLVTVSSAS
SEQ ID NO: 41 (VL amino acid sequence of mAb6 (Humanized K2 12E12))
DIQMTQSPSSLSASVGDRVTITCSASQGISNYLNWYQQKPGKAVKLLIYYTSILHSGVPSRESGSGSGTD
YTLTISSLQPEDFATYYCQQFNKLPPTFGGGTK

In some embodiments, the anti-CD40 antibody is a CD40 agonist antibody. CD40 agonist antibodies are described in WO2010/009346, WO2010/104747 and WO2010/104749. Other anti-CD40 agonist antibodies in development include CP-870,893 that is a fully human IgG2 CD40 agonist antibody developed by Pfizer. It binds CD40 with a KD of 3.48×10-10 M, but does not block binding of CD40L (see e.g., U.S. Pat. No. 7,338,660) and SGN-40 that is a humanized IgG1 antibody developed by Seattle Genetics from mouse antibody clone S2C6, which was generated using a human bladder carcinoma cell line as the immunogen. It binds to CD40 with a KD of 1.0×10⁻⁹ M and works through enhancing the interaction between CD40 and CD40L, thus exhibiting a partial agonist effect (Francisco J A, et al., Cancer Res, 60: 3225-31, 2000). Even more particularly, the CD40 agonist antibody is selected from the group consisting of selected mAb1, mAb2, mAb3, mAb4, mAb5 and mAb6 as described in Table A.

In some embodiments, the heavy chain or the light chain of the CD40 agonist antibody (i.e., the chain that is not conjugated or fused to the Ct antigenic fusion protein) is conjugated or fused to a CD40 binding domain of CD40L (SEQ ID NO:1).

In some embodiments, the CD40 binding domain of CD40L is fused to the C-terminus of a light or heavy chain of said CD40 agonist antibody, optionally via a linker, preferably the FlexV1 linker as described herein after.

In some embodiments, the antibody of the present invention consists of a CD40 agonist antibody wherein the heavy chain of the antibody is fused or conjugated to the Ct antigenic fusion protein and the light chain is conjugated or fused to the CD40 binding domain of CD40L (SEQ ID NO:1).

In some embodiments, the antibody is specific for Langerin. In some embodiments, the antibody derives from the antibody 15B10 having ATCC Accession No. PTA-9852. In some embodiments, the antibody derives from the antibody 2G3 having ATCC Accession No. PTA-9853. In some embodiments, the antibody derives from the antibody 91E7, 37C1, or 4C7 as described in WO2011032161.

In some embodiments, the anti-Langerin antibody comprises a heavy chain comprising the complementarity determining regions CDR1H, CDR2H and CDR3H of the 15B10 antibody and a light chain comprising the complementarity determining regions CDR1L, CDR2L and CDR3L of the 15B10 antibody.

In some embodiments, the anti-Langerin antibody comprises a heavy chain comprising the complementarity determining regions CDR1H, CDR2H and CDR3H of the 2G3 antibody and a light chain comprising the complementarity determining regions CDR1L, CDR2L and CDR3L of the 2G3 antibody.

In some embodiments, the anti-Langerin antibody comprises a heavy chain comprising the complementarity determining regions CDR1H, CDR2H and CDR3H of the 4C7 antibody and a light chain comprising the complementarity determining regions CDR1L, CDR2L and CDR3L of the 4C7 antibody.

In some embodiments, the antibody is selected from the group consisting of selected mAb7, mAb8, mAb9, as described in Table B.

In some embodiments, the antibody is selected from the group consisting of:
an antibody comprising a heavy chain having an amino sequence as set forth in SEQ ID NO:42 and a light chain having an amino sequence as set forth in SEQ ID NO:43 (mAb7);
an antibody comprising a heavy chain having an amino sequence as set forth in SEQ ID NO:44 and a light chain having an amino sequence as set forth in SEQ ID NO:45 (mAb8); and
an anti-CD40 antibody comprising a heavy chain having an amino sequence as set forth in SEQ ID NO:46 and a light chain having an amino sequence as set forth in SEQ ID NO:47 (mAb9).

| | | |
|---|---|---|
| mAb7 [15B10] | SEQ ID NO: 42 | SEQ ID NO: 43 |
| mAb8 [2G3] | SEQ ID NO: 44 | SEQ ID NO: 45 |
| mAb9 [4C7] | SEQ ID NO: 46 | SEQ ID NO: 47 |

SEQ ID NO: 42 (Amino acid sequence of variable
heavy chain region (VH) of 15B10)
QVQLRQSGPELVKPGASVKMSCKASGYTFTDYVISWVKQRTGQGLEWIGD
IYPGSGYSFYNENFKGKATLTADKSSTTAYMQLSSLTSEDSAVYFCATYY
NYPFAYWGQGTLVTVSAAS
SEQ ID NO: 43 (Amino acid sequence of variable
light chain (VL) of 15B10)
DVVMTQTPLSLPVRLGDQASISCRSSQSLVHSNGNTYLHWYLQKPGQSPK
LLIYKVSNRFSGVPDRESGSGSGTNFTLKISRVEAEDLGLYFCSQSTHVP
YTFGGGTK
SEQ ID NO: 44 (Amino acid sequence of variable
heavy chain region (VH) of 2G3)
EVQLVESGGGLVQPKGSLKLSCAASGLTFNIYAMNWVRQAPGKGLEWVAR
IRNKSNNYATYYADSVKDRFTISRDDSQSLLYLQMNNLKTEDTAMYYCVG
RDWFDYWGQGTLVTVSAAS
SEQ ID NO: 45 (Amino acid sequence of variable
light chain (VL) of 2G3)
QAVVTQESALTTSPGETVTLTCRSSTGAVTTSNYANWVQEKPDHLFTGLI
GGTNNRVSGVPARFSGSLIGDKAALTITGAQTEDEAIYFCALWYSNHWVF
GGGTK
SEQ ID NO: 46 (Amino acid sequence of variable
heavy chain region (VH) of 4C7)
QVQLQQSGAELVRPGASVTLSCKASGYTFIDHDMHWVQQTPVYGLEWIGA
IDPETGDTGYNQKFKGKAILTADKSSRTAYMELRSLTSEDSAVYYCTIPF
YYSNYSPFAYWGQGTLVTVSAAKS
SEQ ID NO: 47 (Amino acid sequence of variable
light chain (VL) of 4C7)
QIVLSQSPAILSASPGEKVTMTCRASSSVSYMHWYQRKPGSSPKPWIYAT
SNLASGVPARFSGSGSGTSYSLTISRVEAEDAATYYCQQWSSNPLTFGAG
TK

The antibodies of the invention may be produced by any technique known per se in the art, such as, without limitation, any chemical, biological, genetic or enzymatic technique, either alone or in combination. Knowing the amino acid sequence of the desired sequence, one skilled in the art can readily produce said polypeptides, by standard techniques for production of polypeptides. For instance, the antibodies of the invention can be synthesized by recombinant DNA techniques as is now well-known in the art. For example, these fragments can be obtained as DNA expression products after incorporation of DNA sequences encoding the desired (poly) peptide into expression vectors and introduction of such vectors into suitable eukaryotic or prokaryotic hosts that will express the desired polypeptide, from which they can be later isolated using well-known techniques.

The heavy chain and/or the light of the antibody is conjugated or fused to the Ct antigenic fusion protein via its c-terminus. In some embodiments, the heavy chain and/or the light chain of the antibody is fused to the N-terminus of the Ct antigenic fusion protein.

In some embodiments, the heavy chain and/or the light chain of the antibody is conjugated to the Ct antigenic fusion protein by using chemical coupling. Several methods are known in the art for the attachment or conjugation of an antibody to its conjugate moiety. Examples of linker types that have been used to conjugate a moiety to an antibody include, but are not limited to, hydrazones, thioethers, esters, disulfides and peptide-containing linkers, such as valine-citruline linker. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D). Techniques for conjugating polypeptides and in particular, are well-known in the art (See, e.g., Amon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58; see also, e.g., PCT publication WO 89/12624.) Typically, the peptide is covalently attached to lysine or cysteine residues on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Halder, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106; Junutula, J. R., Flagella, K. M., Graham, R. A., Parsons, K. L., Ha, E., Raab, H., Bhakta, S., Nguyen, T., Dugger, D. L., Li, G., et al. (2010). Engineered thio-trastuzumab-DM1 conjugate with an improved therapeutic index to target human epidermal growth factor receptor 2-positive breast cancer. Clin. Cancer Res. 16, 4769-4778). Junutula et al. (Nat Biotechnol. 2008; 26:925-32) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, substitution, or mutation on the polypeptide). Then a transglutaminase can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (WO 2012059882).

In some embodiments, the heavy chain and/or the light chain of the antibody is conjugated to the Ct antigenic fusion protein by a dockerin domain or multiple domains to permit non-covalent coupling to cohesin fusion proteins as described in US20160031988A1 and US20120039916A1. In some embodiments, the heavy chain and/or the light chain is conjugated via a dockerin domain to the cohesin fusion protein that consists of the amino acid sequence as set forth in SEQ ID NO.48.

> cohesin fusion protein
SEQ ID NO: 48
MDPKGSLSWRILLFLSLAFELSYGGLNDIFEAQKIEWHEDDLDAVRIKVD
TVNAKPGDTVRIPVRESGIPSKGIANCDFVYSYDPNVLEIIEIEPGDIIV
DPNPDKSFDTAVYPDRKIIVFLFAEDSGTGAYAITKDGVFATIVAKVKEG
APNGLSVIKFVEVGGFANNDLVEQKTQFFDGGVNVGDTTEPATPTTPVTT
PTTTDDLDAQTPTNTISVTPTNNSTPTNNSNPKPNPASIDYHEWQASLAL
SYRLNMFTPYIGVKWSSSVSPTTSVHPTPTSVPPTPTKSSPPAANQLITL
SNLHLSLSSLLANNAVTNPPTPTSTPADSSTITPTATPTATPTIKGGKFT
VTPKSSGSMFLVSADIIASRMEGGVVTVTPTATATPSAIVTTITPTATTK
PEKPPKIFILCMTRVPTIDASAMHALEEFFLINGSITVAATAPTVTPTVN
ATPSAACNGVTESFSHSLAPYRALLTFFANSKPSGE

In some embodiments, the heavy chain and/or the light chain of the antibody is fused to the Ct antigenic fusion protein. In some embodiments, the Ct antigenic fusion protein is fused either directly or via a linker to the heavy chain and/or the light chain. In some embodiments, the Ct antigenic fusion protein that has the formula of VS4 bis-f1-PmpG-f2-PgP3-f3-YchM-f4-NqrC and that consists of the amino acid sequence as set forth in SEQ ID NO:12 is fused either directly or via a linker to the heavy chain and/or the light chain of the antibody. In some embodiments, the Ct antigenic fusion protein that has the formula of VS4 bis-f1-PmpG-f2-PgP3-f3-YchM-f4-NqrC and that consists of the amino acid sequence as set forth in SEQ ID NO:12 is fused either directly or via a linker to the heavy chain of the antibody. In some embodiments, the linker is the FlexV1 linker as set forth in SEQ ID NO:7.

Nucleic Acids, Vectors and Host Cells of the Present Invention:

A further object of the present invention relates to a nucleic acid that encodes for a Ct antigenic polypeptide of the present invention.

A further object of the present invention relates to a nucleic acid that encodes for a Ct antigenic fusion protein of the present invention.

A further object of the invention relates to a nucleic acid that encodes for a heavy chain and/or a light chain of an antibody directed against a surface antigen of an antigen presenting cell that is fused to the Ct antigenic fusion protein of the present invention.

Typically, said nucleic acid is a DNA or RNA molecule, which may be included in any suitable vector, such as a plasmid, cosmid, episome, artificial chromosome, phage or a viral vector.

So, a further object of the invention relates to a vector comprising a nucleic acid of the present invention.

Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40, LTR promoter and enhancer of Moloney mouse leukemia virus, promoter and enhancer of immunoglobulin H chain and the like. Any expression vector for animal cell can be used, so long as a gene encoding the human antibody C region can be inserted and expressed. Examples of suitable vectors include pAGE107, pAGE103, pHSG274, pKCR, pSG1 beta d2-4 and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A further object of the present invention relates to a host cell which has been transfected, infected or transformed by a nucleic acid and/or a vector according to the invention.

The nucleic acids of the invention may be used to produce the polypeptides of the present invention in a suitable expression system. Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts. Mammalian host cells include Chinese Hamster Ovary (CHO cells) including dhfr-CHO cells (described in Urlaub and Chasin, 1980) used with a DHFR selectable marker, CHOK1 dhfr+ cell lines, NSO myeloma cells, COS cells and SP2 cells, for example GS CHO cell lines together with GS Xceed™ gene expression system (Lonza), or HEK cells.

The present invention also relates to a method of producing a recombinant host cell expressing a polypeptide according to the invention, said method comprising the steps of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody. Such recombinant host cells can be used for the production of polypeptides of the present invention.

The host cell as disclosed herein are thus particularly suitable for producing the polypeptides of the present invention. Indeed, when recombinant expression are introduced into mammalian host cells, the polypeptides are produced by culturing the host cells for a period of time sufficient for expression of the polypeptide in the host cells and, optionally, secretion of the polypeptide into the culture medium in which the host cells are grown. The polypeptides can be recovered and purified for example from the culture medium after their secretion using standard protein purification methods.

Pharmaceutical and Vaccine Compositions:

The Ct antigenic polypeptides, the Ct antigenic fusion proteins as well as antibodies as described herein may be administered as part of one or more pharmaceutical compositions. Except insofar as any conventional carrier medium is incompatible with the Ct antigenic polypeptides, the Ct antigenic fusion proteins as well as antibodies, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The Ct antigenic polypeptides, the Ct antigenic fusion proteins as well as the antibodies as described herein are particularly suitable for preparing vaccine composition.

Thus a further object of the present invention relates to a vaccine composition comprising one or more Ct antigenic polypeptides, one or more Ct antigenic fusion proteins or one more antibodies of the present invention.

In some embodiments, the vaccine composition of the present invention comprises an adjuvant. In some embodiments, the adjuvant is alum. In some embodiments, the adjuvant is Incomplete Freund's adjuvant (IFA) or other oil based adjuvant that is present between 30-70%, preferably between 40-60%, more preferably between 45-55% proportion weight by weight (w/w). In some embodiments, the vaccine composition of the present invention comprises at least one Toll-Like Receptor (TLR) agonist which is selected from the group consisting of TLR1, TLR2, TLR3, TLR4, TLR5, TLR6, TLR7, and TLR8 agonists.

Therapeutic Methods:

The pharmaceutical or vaccine compositions as herein described are particularly suitable for inducing an immune response against Chlamydia trachomatis and thus can be used for vaccine purposes.

Therefore, a further object of the present invention relates to a method for vaccinating a subject in need thereof against Chlamydia trachomatis comprising administering a therapeutically effective amount of a pharmaceutical or vaccine composition of the present invention.

In some embodiments, the vaccine compositions as herein described are particularly suitable for the treatment of Trachoma.

In some embodiments, the subject can be human or any other animal (e.g., birds and mammals) susceptible to Chlamydia infection (e.g., domestic animals such as cats and dogs; livestock and farm animals such as horses, cows, pigs, chickens, etc.). Typically said subject is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, the subject is a non-human animal. In some embodiments, the subject is a farm animal or pet. In some embodiments, the subject is a human. In some embodiments, the subject is a human infant. In some embodiments, the subject is a human child. In some embodiments, the subject is a human adult. In some embodiments, the subject is an elderly human. In some embodiments, the subject is a premature human infant.

In some embodiments, the subject can be symptomatic or asymptomatic.

Typically, the active ingredient of the present invention (i.e., the pharmaceutical or vaccine composition) is administered to the subject at a therapeutically effective amount. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; the activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. In particular, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, in particular from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The pharmaceutical or vaccine compositions as herein described may be administered to the subject by any route of administration and in particular by oral, nasal, rectal, topical, buccal (e.g., sub-lingual), parenteral (e.g., subcutaneous, intramuscular, intradermal, or intravenous) and transdermal administration, although the most suitable route in any given case will depend on the nature and severity of the condition being treated and on the nature of the particular active agent which is being used.

The invention will be further illustrated by the following figures and examples. However, these examples and figures should not be interpreted in any way as limiting the scope of the present invention.

### FIGURES

FIG. 1. Schematic representation of the first polyepitopic Chlamydia DC vaccine generated. Selected antigens from C. Trachomatis, chosen from the literature and our in silico analyses are fused with the heavy chain of the αCD40 mAb, while adding flexible spacers to improve their synthesis and/or secretion.

FIG. 2: In vitro expansion of C. trachomatis Ct-5pp-specific memory T-cells by the αCD40 vaccine construct. PBMCs of 4 donors were stimulated with the αCD40 mAb associated with the Ct-5pp antigens or Ebola GP as irrelevant control (EBOV) and were then restimulated at day 9 with pools of peptides overlapping Ct-5pp. The percentages of CD4+ T cells producing any of tested cytokines after 10 days of culture are shown.

### EXAMPLE 1

We have identified specific epitopes to be included in vaccine candidates against Chlamydia trachomatis thanks to in silico analysis of the amino-acid sequence of MOMP VS4, YchM, PgP3, PmPG and NqrC proteins to map predicted MHC-I and -II epitopes by online software and peptide binding prediction software. The five proteins were analysed using NetMHC 4.0 (https://services.healthtech.dtu.dk/service.php?NetMHC-4.0) and NetMHCII 2.3 (https://services.healthtech.dtu.dk/service.php?NetMHCII-2.3), MHC class-I and MHC class-II/peptide binding prediction softwares, respectively. Linear B-cell epitopes were predicted using BepiPred 2.0 (https://services.healthtech.dtu.dk/service.php?BepiPred-2.0). Based on the above mentioned methodology we have identified the following epitopes of interest:

| | VS4 bis |
|---|---|
| SEQ ID NO: 2 | |
| | ASIDYHEWQASLALSYRLNMFTPYIGVKWS |
| | YchM antigen |
| SEQ ID NO: 3 | |
| | EKPPKIFILCMTRVPTIDASAMHALEEFFL |
| | PgP3 antigen |
| SEQ ID NO: 4 | |
| | GKFTVTPKSSGSMFLVSADIIASRMEGGVV |
| | PmPG antigen |
| SEQ ID NO: 5 | |
| | PAANQLITLSNLHLSLSSLLANNAVTNPPT |
| | NqrC antigen |
| SEQ ID NO: 6 | |
| | CNGVTESFSHSLAPYRALLTFFANSKPSGE |

### EXAMPLE 2

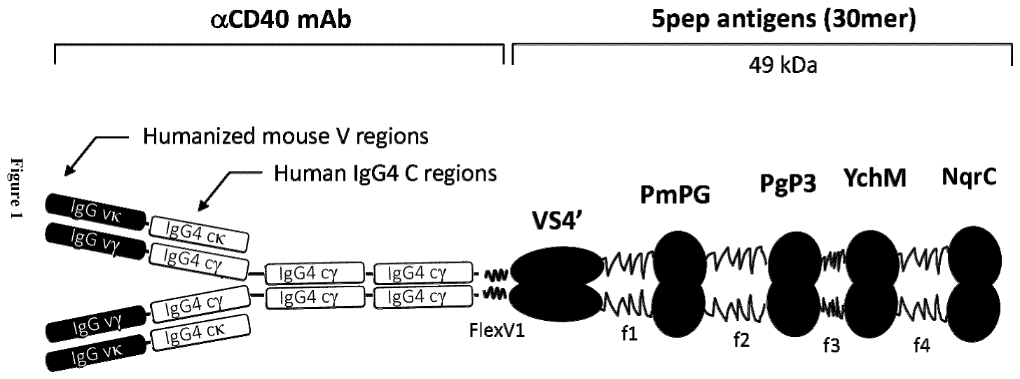

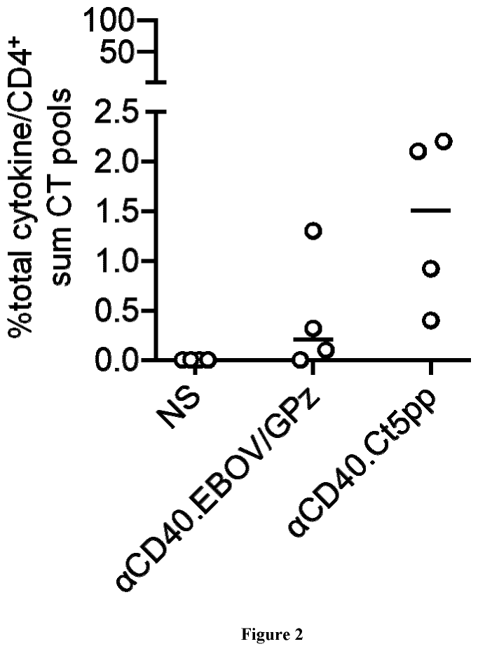

The polyepitope protein of SEQ ID NO:12 was prepared and conjugated to an anti-CD40 antibody according to FIG. 1. PBMCs from Chlamydia infected individuals were keeping frozen. After thawing, cells are cultured in RPMI supplemented with 10% human serum (SAB, Jacques boys) for overnight resting. Cells were then cultured in the presence of 10 nM of αCD40.Ct-5pp or irrelevant antigen. Non stimulated or SEB (10 ng/ul) stimulated cells were used as negative and positive controls respectively. At day 2 and 5 of culture, medium was refreshed with supplement of IL-2 (100U/mL). At day 7, medium was renewed and cells kept for resting 24H without any cytokines. On day 8, cells were stimulated in the presence of brefeldin A (5ug/ml) by pools of overlapping peptides covering each antigen of the polyepitopic Chlamydia construct. Cells were subsequently stained for phenotypical markers (CD3, CD4, CD8) and for their intracellular synthesize of cytokines during peptide stimulation (IFNg, TNFa, MIP-1b, IL-2, IL-4, IL-13, IL-10). Fluorescence of PFA-fixed cells was assessed by flow cytometry using BD LSRII flow cytometer. Data were subsequently analyzed by FlowJo software (Tristar). The results are depicted in FIG. 2.

### REFERENCES

Throughout this application, various references describe the state of the art to which this invention pertains. The disclosures of these references are hereby incorporated by reference into the present disclosure.

1.-33. (canceled)

34. A Chlamydia trachomatis (Ct) antigenic polypeptide that comprises:
a VS4 bis polypeptide having an amino acid sequence having at least 80% of identity with the amino acid as set forth in SEQ ID NO:2, or
a YchM polypeptide having an amino acid sequence having at least 80% of identity with the amino acid as set forth in SEQ ID NO:3, or
a PgP3 polypeptide having an amino acid sequence having at least 80% of identity with the amino acid as set forth in SEQ ID NO:4, or
a PmPG polypeptide having an amino acid sequence having at least 80% of identity with the amino acid as set forth in SEQ ID NO:5, or
an NqrC polypeptide having an amino acid sequence having at least 80% of identity with the amino acid as set forth in SEQ ID N0:6.

35. A Ct antigenic fusion protein that comprises one or more Ct antigenic polypeptide(s) selected from:
a VS4 bis polypeptide having an amino acid sequence having at least 80% of identity with the amino acid as set forth in SEQ ID NO:2,
a YchM polypeptide having an amino acid sequence having at least 80% of identity with the amino acid as set forth in SEQ ID NO:3,
a PgP3 polypeptide having an amino acid sequence having at least 80% of identity with the amino acid as set forth in SEQ ID NO:4,
a PmPG polypeptide having an amino acid sequence having at least 80% of identity with the amino acid as set forth in SEQ ID NO:5, and
a NqrC polypeptide having an amino acid sequence having at least 80% of identity with the amino acid as set forth in SEQ ID NO:6,
optionally fused to each other directly or via a linker selected from the group consisting of FlexV1, f1, f2, f3, or f4.

36. The Ct antigenic fusion protein of claim 35 that has the formula of (Ag-L)n
wherein
Ag represents a Ct antigenic polypeptide that comprises:
a VS4 bis polypeptide having an amino acid sequence having at least 80% of identity with the amino acid as set forth in SEQ ID NO:2, or
a YchM polypeptide having an amino acid sequence having at least 80% of identity with the amino acid as set forth in SEQ ID NO:3, or
a PgP3 polypeptide having an amino acid sequence having at least 80% of identity with the amino acid as set forth in SEQ ID NO:4, or
a PmPG polypeptide having an amino acid sequence having at least 80% of identity with the amino acid as set forth in SEQ ID NO:5, or
an NqrC polypeptide having an amino acid sequence having at least 80% of identity with the amino acid as set forth in SEQ ID NO:6,
L represents a linker, and
n represents an integer number from 1 to 5.

37. The Ct antigenic fusion protein of claim 35 that comprises in said order the VS4 bis antigenic polypeptide, the PmpG antigenic polypeptide, the PgP3 antigenic polypeptide, the YchM antigenic polypeptide, and the NqrC antigenic polypeptide.

38. The Ct antigenic fusion protein of claim 35 that has the formula of VS4 bis-f1-PmpG-f2-PgP3-f3-YchM-f4-NqrC and that consists of the amino acid sequence as set forth in SEQ ID NO:12.

39. An antibody that is directed against a surface antigen of an antigen presenting cell wherein the heavy chain and/or the light chain is conjugated or fused to the Ct antigenic fusion protein of claim 35.

40. The antibody of claim 39, wherein the heavy chain of the antibody and/or the light chain of the antibody is conjugated or fused to a Ct antigenic fusion protein that comprises one or more Ct antigenic polypeptide(s) selected from:
a VS4 bis polypeptide having an amino acid sequence having at least 80% of identity with the amino acid as set forth in SEQ ID NO:2,
a YchM polypeptide having an amino acid sequence having at least 80% of identity with the amino acid as set forth in SEQ ID NO:3,
a PgP3 polypeptide having an amino acid sequence having at least 80% of identity with the amino acid as set forth in SEQ ID NO:4,
a PmPG polypeptide having an amino acid sequence having at least 80% of identity with the amino acid as set forth in SEQ ID NO:5, and
a NqrC polypeptide having an amino acid sequence having at least 80% of identity with the amino acid as set forth in SEQ ID NO:6,
optionally fused to each other directly or via a linker selected from the group consisting of FlexV1, f1, f2, f3, or f4.

41. The antibody of claim 39 that is an IgG antibody.

42. The antibody of claim 39 that is specific for CD40, and wherein the antibody
derives from the 12E12 antibody and comprises:
(a) a heavy chain comprising the complementarity determining regions CDR1H, CDR2H and CDR3H, the CDR1H having the amino acid sequence GFTFSDYYMY (SEQ ID NO:13), the CDR2H having the amino acid sequence YINSGGGSTYYPDTVKG (SEQ ID NO:14), and the CDR3H having the amino acid sequence RGLPFHAMDY (SEQ ID NO:15),
(b) and a light chain comprising the complementarity determining regions CDR1L, CDR2L and CDR3L, the CDR1L having the amino acid sequence SASQGISNYLN (SEQ ID NO:16) the CDR2L having the amino acid sequence YTSILHS (SEQ ID NO:17) and the CDR3L having the amino acid sequence QQFNKLPPT (SEQ ID NO:18)
or derives from the 11B6 antibody and comprises:
(c) a heavy chain comprising the complementarity determining regions CDR1H, CDR2H and CDR3H, the CDR1H having the amino acid sequence GYSFTGYYMH (SEQ ID NO:19), the CDR2H having the amino acid sequence RINPYNGATSYNQNFKD (SEQ ID NO:20), and the CDR3H having the amino acid sequence EDYVY (SEQ ID NO:21), and
(d) a light chain comprising the complementarity determining regions CDR1L, CDR2L and CDR3L, the CDR1L having the amino acid sequence RSSQSLVHSNGNTYLH (SEQ ID NO:22) the CDR2L having the amino acid sequence KVSNRFS (SEQ ID NO:23) and the CDR3L having the amino acid sequence SQSTHVPWT (SEQ ID NO:24)
or derives from the 12B4 antibody and comprises:
(e) a heavy chain comprising the complementarity determining regions CDR1H, CDR2H and CDR3H, the CDR1H having the amino acid sequence GYTFTDYVLH (SEQ ID NO:25), the CDR2H having the amino acid sequence YINPYNDGTKYNEKFKG (SEQ ID NO:26), and the CDR3H having the amino acid sequence GYPAYSGYAMDY (SEQ ID NO:27), and
(f) a light chain comprising the complementarity determining regions CDR1L, CDR2L and CDR3L, the CDR1L having the amino acid sequence RASQDISNYLN (SEQ ID NO:28) the CDR2L having the amino acid sequence YTSRLHS (SEQ ID N0:29) and the CDR3L having the amino acid sequence HHGNTLPWT (SEQ ID NO:30).

43. The antibody of claim 39 that is a CD40 agonist antibody whose heavy chain or the light chain (i.e., the chain that is not conjugated or fused to the Ct antigenic fusion protein) is conjugated or fused to a CD40 binding domain of CD40L (SEQ ID NO:1).

44. The antibody of claim 39 that is a CD40 agonist antibody whose heavy chain or the light chain (i.e., the chain that is not conjugated or fused to the Ct antigenic fusion protein) is conjugated or fused to a CD40 binding domain of CD40L (SEQ ID NO:1), wherein:
the CD40 binding domain of CD40L is fused to the C-terminus of a light or heavy chain of said CD40 agonist antibody, optionally via a linker, or
the heavy chain of the antibody is fused or conjugated to the Ct antigenic fusion protein and the light chain is conjugated or fused to the CD40 binding domain of CD40L (SEQ ID NO:1).

45. The antibody of claim 39 that is specific for Langerin and that comprises:
a heavy chain comprising the complementarity determining regions CDR1H, CDR2H and CDR3H of the 15B10 antibody and a light chain comprising the complementarity determining regions CDR1L, CDR2L and CDR3L of the 15B10 antibody, or
a heavy chain comprising the complementarity determining regions CDR1H, CDR2H and CDR3H of the 2G3 antibody and a light chain comprising the complementarity determining regions CDR1L, CDR2L and CDR3L of the 2G3 antibody, or
a heavy chain comprising the complementarity determining regions CDR1H, CDR2H and CDR3H of the 4C7 antibody and a light chain comprising the complementarity determining regions CDR1L, CDR2L and CDR3L of the 4C7 antibody.

46. The antibody of claim 39 that comprises a heavy chain or light chain that is conjugated via a dockerin domain to the cohesin fusion protein that consists of the amino acid sequence as set forth in SEQ ID NO:48.

47. The antibody of claim 39, wherein the heavy chain and/or the light chain is fused to a Ct antigenic fusion protein that comprises one or more Ct antigenic polypeptide(s) selected from:
a VS4 bis polypeptide having an amino acid sequence having at least 80% of identity with the amino acid as set forth in SEQ ID NO:2,
a YchM polypeptide having an amino acid sequence having at least 80% of identity with the amino acid as set forth in SEQ ID NO:3,
a PgP3 polypeptide having an amino acid sequence having at least 80% of identity with the amino acid as set forth in SEQ ID NO:4,
a PmPG polypeptide having an amino acid sequence having at least 80% of identity with the amino acid as set forth in SEQ ID NO:5, and
a NqrC polypeptide having an amino acid sequence having at least 80% of identity with the amino acid as set forth in SEQ ID NO:6,
optionally fused to each other directly or via a linker selected from the group consisting of FlexV1, f1, f2, f3, or f4
via the linker selected from the group consisting of FlexV1, f1, f2, f3, and f4.

48. A nucleic acid that encodes for the heavy chain and/or the light chain of the antibody of claim 39.

49. A vector comprising the nucleic acid of claim 48.

50. A host cell which has been transfected, infected, or transformed by the nucleic acid of claim 48.

51. A host cell which has been transfected, infected, or transformed by the vector of claim 49.

52. A vaccine composition that comprises one or more antibody(ies) of claim 39.

53. A method for vaccinating a subject in need thereof against Chlamydia trachomatis comprising administering a therapeutically effective amount of one or more antibody(ies) of claim 39.


SEQUENCE LISTING (48 sequences)

SEQ ID NO: 1 - protein, 149 aa, Artificial
  CD40L binding domain
MQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 2 - protein, 30 aa, Artificial
  VS4 bis antigen
ASIDYHEWQASLALSYRLNMFTPYIGVKWS

SEQ ID NO: 3 - protein, 30 aa, Artificial
  YchM antigen
EKPPKIFILCMTRVPTIDASAMHALEEFFL

SEQ ID NO: 4 - protein, 30 aa, Artificial
  PgP3 antigen
GKFTVTPKSSGSMFLVSADIIASRMEGGVV

SEQ ID NO: 5 - protein, 30 aa, Artificial
  PmPG antigen
PAANQLITLSNLHLSLSSLLANNAVTNPPT

SEQ ID NO: 6 - protein, 30 aa, Artificial
  NqrC antigen
CNGVTESFSHSLAPYRALLTFFANSKPSGE

SEQ ID NO: 7 - protein, 27 aa, Artificial
  linker
QTPTNTISVTPTNNSTPTNNSNPKPNP

SEQ ID NO: 8 - protein, 25 aa, Artificial
  linker
SSVSPTTSVHPTPTSVPPTPTKSSP

SEQ ID NO: 9 - protein, 25 aa, Artificial
  linker
PTSTPADSSTITPTATPTATPTIKG

SEQ ID NO: 10 - protein, 25 aa, Artificial
  linker
TVTPTATATPSAIVTTITPTATTKP

SEQ ID NO: 11 - protein, 25 aa, Artificial
  linker
TNGSITVAATAPTVTPTVNATPSAA

SEQ ID NO: 12 - protein, 250 aa, Artificial
  fusion protein
ASIDYHEWQASLALSYRLNMFTPYIGVKWSSSVSPTTSVHPTPTSVPPTPTKSSPPAANQLITLSNLHLSLSSLLANNAVTNPPTPTSTPADSSTITPTATPTATPTIKGGKFTVTPKSSGSMFLVSADIIASRMEGGVVTVTPTATATPSAIVTTITPTATTKPEKPPKIFILCMTRVPTIDASAMHALEEFFLTNGSITVAATAPTVTPTVNATPSAACNGVTESFSHSLAPYRALLTFFANSKPSGE

SEQ ID NO: 13 - protein, 10 aa, Artificial
  H-CDR1
GFTFSDYYMY

SEQ ID NO: 14 - protein, 17 aa, Artificial
  H-CDR2
YINSGGGSTYYPDTVKG

SEQ ID NO: 15 - protein, 10 aa, Artificial
  H-CDR3
RGLPFHAMDY

SEQ ID NO: 16 - protein, 11 aa, Artificial
  L-CDR1
SASQGISNYLN

SEQ ID NO: 17 - protein, 7 aa, Artificial
  L-CDR2
YTSILHS

SEQ ID NO: 18 - protein, 9 aa, Artificial
  L-CDR3
QQFNKLPPT

SEQ ID NO: 19 - protein, 10 aa, Artificial
  H-CDR1
GYSFTGYYMH

SEQ ID NO: 20 - protein, 17 aa, Artificial
  H-CDR2
RINPYNGATSYNQNFKD

SEQ ID NO: 21 - protein, 5 aa, Artificial
  H-CDR3
EDYVY

SEQ ID NO: 22 - protein, 16 aa, Artificial
  L-CDR1
RSSQSLVHSNGNTYLH

SEQ ID NO: 23 - protein, 7 aa, Artificial
  L-CDR2
KVSNRFS

SEQ ID NO: 24 - protein, 9 aa, Artificial
  L-CDR3
SQSTHVPWT

SEQ ID NO: 25 - protein, 10 aa, Artificial
  H-CDR1
GYTFTDYVLH

SEQ ID NO: 26 - protein, 17 aa, Artificial
  H-CDR2
YINPYNDGTKYNEKFKG

SEQ ID NO: 27 - protein, 12 aa, Artificial
  H-CDR3
GYPAYSGYAMDY

SEQ ID NO: 28 - protein, 11 aa, Artificial
  L-CDR1
RASQDISNYLN

SEQ ID NO: 29 - protein, 7 aa, Artificial
  L-CDR2
YTSRLHS

SEQ ID NO: 30 - protein, 9 aa, Artificial
  L-CDR3
HHGNTLPWT

SEQ ID NO: 31 - protein, 116 aa, Artificial
  VH domain
EVQLVQSGAEVKKPGASVKISCKASGYSFTGYYMHWVKQAHGQGLEWIGRINPYNGATSYNQNFKDRATLTVDKSTSTAYMELSSLRSEDTAVYYCAREDYVYWGQGTTVTVSSAS

SEQ ID NO: 32 - protein, 108 aa, Artificial
  VL domain
DVVMTQSPLSLPVTLGQPASISCRSSQSLVHSNGNTYLHWYQQRPGQSPRLLIYKVSNRFSGVPDRFSGSGSGTDFTLKISRVEAEDVGVYFCSQSTHVPWTFGGGTK

SEQ ID NO: 33 - protein, 116 aa, Artificial
  VH domain
EVQLVQSGAEVKKPGASVKVSCKASGYSFTGYYMHWVRQAPGQGLEWIGRINPYNGATSYNQNFKDRVTLTVDKSTSTAYMELSSLRSEDTAVYYCAREDYVYWGQGTTVTVSSAS

SEQ ID NO: 34 - protein, 123 aa, Artificial
  VH domain
EVQLQQSGPELVKPGASVKMSCKASGYTFTDYVLHWVKQKPGQGLEWIGYINPYNDGTKYNEKFKGKATLTSDKSSSTAYMELSSLTSEDSAVYYCARGYPAYSGYAMDYWGQGTSVTVSSAS

SEQ ID NO: 35 - protein, 103 aa, Artificial
  VL domain
DIQMTQTTSSLSASLGDRVTISCRASQDISNYLNWYQQKPDGTVKLLIYYTSRLHSGVPSRFSGSGSGTDYSLTISNLEQEDIATYFCHHGNTLPWTFGGGTK

SEQ ID NO: 36 - protein, 121 aa, Artificial
  VH domain
DVQLQESGPDLVKPSQSLSLTCTVTGYSITSDYSWHWIRQFPGNKLEWMGYIYYSGSTNYNPSLKSRISITRDTSKNQFFLQLNSVTTEDSATYFCARFYYGYSFFDYWGQGTTLTVSSAS

SEQ ID NO: 37 - protein, 102 aa, Artificial
  VL domain
QIVLTQSPAFMSASPGEKVTMTCSASSSVSYMHWYQQKSGTSPKRWIYDTSKLASGVPARFSGSGSGTSYSLTISSMEAEDAATYYCQQWSSNPLTFGAGTK

SEQ ID NO: 38 - protein, 128 aa, Artificial
  VH domain
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSAS

SEQ ID NO: 39 - protein, 103 aa, Artificial
  VL domain
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTK

SEQ ID NO: 40 - protein, 121 aa, Artificial
  VH domain
EVQLVESGGGLVQPGGSLKLSCATSGFTFSDYYMYWVRQAPGKGLEWVAYINSGGGSTYYPDTVKGRFTISRDNAKNTLYLQMNSLRAEDTAVYYCARRGLPFHAMDYWGQGTLVTVSSAS

SEQ ID NO: 41 - protein, 103 aa, Artificial
  VL domain
DIQMTQSPSSLSASVGDRVTITCSASQGISNYLNWYQQKPGKAVKLLIYYTSILHSGVPSRFSGSGSGTDYTLTISSLQPEDFATYYCQQFNKLPPTFGGGTK

SEQ ID NO: 42 - protein, 119 aa, Artificial
  VH domain
QVQLRQSGPELVKPGASVKMSCKASGYTFTDYVISWVKQRTGQGLEWIGDIYPGSGYSFYNENFKGKATLTADKSSTTAYMQLSSLTSEDSAVYFCATYYNYPFAYWGQGTLVTVSAAS

SEQ ID NO: 43 - protein, 108 aa, Artificial
  VL domain
DVVMTQTPLSLPVRLGDQASISCRSSQSLVHSNGNTYLHWYLQKPGQSPKLLIYKVSNRFSGVPDRFSGSGSGTNFTLKISRVEAEDLGLYFCSQSTHVPYTFGGGTK

SEQ ID NO: 44 - protein, 119 aa, Artificial
  VH domain
EVQLVESGGGLVQPKGSLKLSCAASGLTFNIYAMNWVRQAPGKGLEWVARIRNKSNNYATYYADSVKDRFTISRDDSQSLLYLQMNNLKTEDTAMYYCVGRDWFDYWGQGTLVTVSAAS

SEQ ID NO: 45 - protein, 105 aa, Artificial
  VL domain
QAVVTQESALTTSPGETVTLTCRSSTGAVTTSNYANWVQEKPDHLFTGLIGGTNNRVSGVPARFSGSLIGDKAALTITGAQTEDEAIYFCALWYSNHWVFGGGTK

SEQ ID NO: 46 - protein, 124 aa, Artificial
  heavy chain
QVQLQQSGAELVRPGASVTLSCKASGYTFIDHDMHWVQQTPVYGLEWIGAIDPETGDTGYNQKFKGKAILTADKSSRTAYMELRSLTSEDSAVYYCTIPFYYSNYSPFAYWGQGTLVTVSAAKS

SEQ ID NO: 47 - protein, 102 aa, Artificial
  light chain
QIVLSQSPAILSASPGEKVTMTCRASSSVSYMHWYQRKPGSSPKPWIYATSNLASGVPARFSGSGSGTSYSLTISRVEAEDAATYYCQQWSSNPLTFGAGTK

SEQ ID NO: 48 - protein, 486 aa, Artificial
  cohesin fusion protein
MDPKGSLSWRILLFLSLAFELSYGGLNDIFEAQKIEWHEDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDAQTPTNTISVTPTNNSTPTNNSNPKPNPASIDYHEWQASLALSYRLNMFTPYIGVKWSSSVSPTTSVHPTPTSVPPTPTKSSPPAANQLITLSNLHLSLSSLLANNAVTNPPTPTSTPADSSTITPTATPTATPTIKGGKFTVTPKSSGSMFLVSADIIASRMEGGVVTVTPTATATPSAIVTTITPTATTKPEKPPKIFILCMTRVPTIDASAMHALEEFFLTNGSITVAATAPTVTPTVNATPSAACNGVTESFSHSLAPYRALLTFFANSKPSGE